(19) United States
(12) Patent Application Publication
(10) Pub. No.: US 2010/0174097 A1
(43) Pub. Date: Jul. 8, 2010

(54) METHOD FOR ISOLATION OF HIGH CONCENTRATION OF MAGNESIUM LITHOSPERMATE B FROM SALVIAE MILTIORRHIZAE

(76) Inventors: Man Kil Jung, Seoul (KR); Hyun Chul Lee, Seoul (KR)

Correspondence Address: IPLA P.A., 3550 WILSHIRE BLVD., 17TH FLOOR, LOS ANGELES, CA, 90010, US

(21) Appl. No.: 12/279,040

(22) PCT Filed: Mar. 28, 2007
(86) PCT No.: PCT/KR07/01521

§ 371 Date: Aug. 11, 2008

(30) Foreign Application Priority Data
Mar. 28, 2007 (KR) 10-2007-0030228

(51) Int. Cl. C07D 307/78

(52) U.S. Cl. 549/468

(57) ABSTRACT

Disclosed herein are simple and economical methods for the isolation of a high concentration of magnesium lithospermate B from Salviae miltiorrhizae Radix and the production of a Salviae miltiorrhizae extract containing a high concentration of magnesium lithospermate B. Without using expensive separation materials (Sephadex LH-20, MCI gel CHP 20P, silica gel), the methods can isolate magnesium lithospermate B at a yield twice as high as in conventional methods and can be used to purify a Salviae miltiorrhizae extract containing 16 times as much magnesium lithospermate as conventional methods.

### CROSS REFERENCES

This application claims priority of International Application No. PCT/2007/001521 filed 28 Mar. 2007, which claims priority of Korean Patent Application No. 10-2007-0030228 filed 28 Mar. 2007 with the Korean Intellectual Property Office.

### TECHNICAL FIELD

The present invention relates to a method for isolating magnesium lithospermate B. More particularly, the present invention relates to a method for isolating a high concentration of magnesium lithospermate B from Salviae miltiorrhizae.

### BACKGROUND ART

Salviae miltiorrhizae, originally coming from China, is a perennial herb belonging to the Labiatae family. It has purple leaves and the roots are red and taste bitter. In herbal medicine, Salviae miltiorrhizae has been used as an active ingredient of tonics, an emmenagogue, an anodyne, an antiphlogistic, and a hemostatic, and in the treatment of gynecologic disorders, including menstrual irregularity, climacterium, abdominal pain, postpartum pain, rheumatism, etc. In addition, Salviae miltiorrhizae is used to promote blood circulation, to alleviate edema, and to promote antihypertensive, anti-coagulant and antibacterial activity, and has been reported to improve the renal functions of rats suffering from chronic renal failure.

Previously, it was discovered by the present inventors that lithospermate B isolated from Salviae miltiorrhizae, or salts thereof (e.g., magnesium lithospermate B, sodium lithospermate B, ammonium lithospermate B, potassium lithospermate B, calcium lithospermate B, ammonium-potassium lithospermate B, etc.) functions to inhibit the proliferation and migration of vascular smooth muscle cells (VSMC) and is very useful in the prevention and treatment of atherosclerosis (U.S. Pat. Publication No. 2007-0027209). Also, the present inventors found excellent effects of lithospermate B isolated from Salviae miltiorrhizae, or salts thereof (e.g., magnesium lithospermate B, sodium lithospermate B, ammonium lithospermate B, potassium lithospermate B, calcium lithospermate B, ammonium-potassium lithospermate B, etc.) on the prevention and treatment of diabetic nephropathy and microalbuminuria (U.S. Pat. No. 6,267,992). Particularly, the present inventors revealed that magnesium lithospermate B is 2.5 times more effective than epalrestat in inhibiting the activity of aldose reductase and the production of fibronectin on mesangial cells (Mankil Jung et al., “Effective Isolation of Magnesium Lithospermate B and Its Inhibition of Aldose Reductase and Fibronectin on Mesangial Cell Line,” Chem. Pharm. Bull. 50(8) 1135-1136 (2002)]).

Conventionally, the isolation of lithospermate or salts thereof (e.g., magnesium lithospermate B) from Salviae miltiorrhizae Radix or the purification of a Salviae miltiorrhizae extract containing such an active ingredient is usually achieved through column chromatography using an adsorbent (stationary phase), such as Sephadex LH-20, MCI gel CHP 20P or silica gel (Takashi Tanaka, et al., [“Magnesium and Ammonium-Potassium Lithospermate B, the Active Principles Having a Uremia-Preventive Effect from Salviae miltiorrhizae,” Chem. Pharm. Bull. 37(2) 340-344 (1989)]; and Mankil Jung, et al., [“Effective Isolation of Magnesium Lithospermate B and Its Inhibition of Aldose Reductase and Fibronectin on Mesangial Cell Line,” Chem. Pharm. Bull. 50(8) 1135-1136 (2002)]). The isolate obtained through column chromatography is, however, low in magnesium lithospermate B content. Further, Sephadex LH-20, MCI gel CHP 20P, or silica gel, which are used as adsorbents (stationary phase), are moderately or very expensive, and thus the chromatographic method is not economically desirable. On the other hand, Korean Pat. Publication No. 2006-011857 discloses a method for preparing a Salviae miltiorrhizae extract using a polar solvent (water, ethanol, buthanol or an admixture thereof) or a non-polar solvent (ethyl acetate, hexane, chloroform, etc.). However, this method affords only a low yield of the Salviae miltiorrhizae extract. Nowhere is the concentration of the active ingredient (e.g., magnesium lithospermate B) in the extract mentioned in Korean Pat. Publication No. 2006-011857.

Leading to the present invention, intensive and thorough research on the isolation of magnesium lithospermate B from Salviae miltiorrhizae radix, conducted by the present inventors, resulted in the finding that the use of a combination of an aqueous solvent and an organic solvent improves the isolation yield of magnesium lithospermate B.

### DISCLOSURE OF INVENTION

It is an object of the present invention to provide a method for isolating magnesium lithospermate B (having the following chemical formula 1) from Salviae miltiorrhizae and purifying a Salviae miltiorrhizae extract containing a high concentration of magnesium, easily and economically.

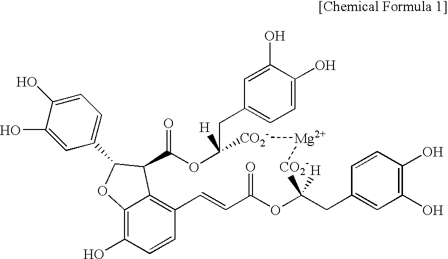

The methods according to the present invention can produce a Salviae miltiorrhizae extract containing magnesium lithospermate B (MLB), which is an active ingredient highly useful in the treatment and prevention of diabetic nephropathy, microalbuminuria and uremia, easily and economically on a mass scale.

### BRIEF DESCRIPTION OF THE DRAWINGS

FIG. 1 is a flow chart illustrating the process of isolating a high concentration of magnesium lithospermate B from Salviae miltiorrhizae Radix in accordance with Example 1 of the present invention.

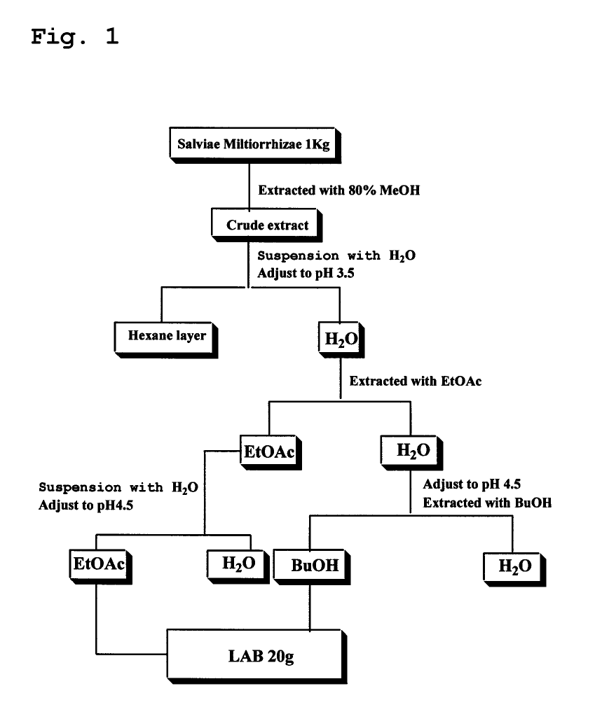

FIG. 2 is a flow chart illustrating the process of producing a Salviae miltiorrhizae extract containing a high concentration of magnesium lithospermate B in accordance with Example 2 of the present invention.

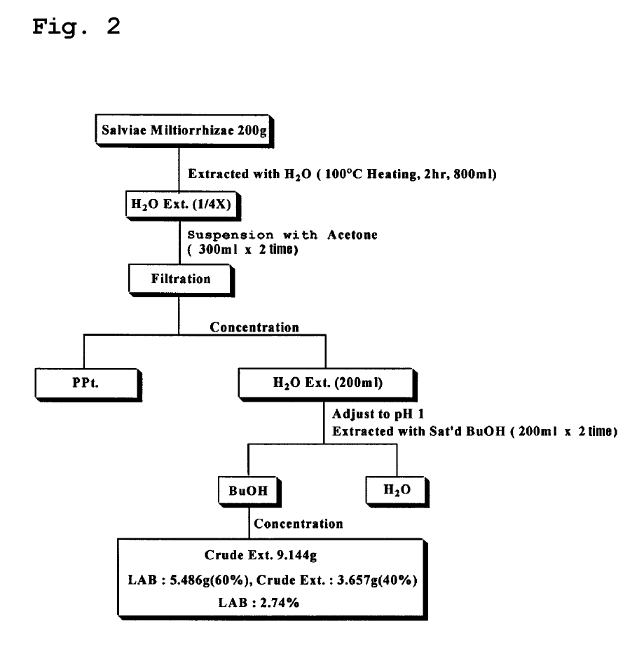

### BEST MODE FOR CARRYING OUT THE INVENTION

In accordance with the present invention, a simple and economical method is provided for isolating magnesium lithospermate B from Salviae miltiorrhizae, comprising (a) finely cutting roots of Salviae miltiorrhizae into sections 1 cm in size and immersing the sections in 80% methanol at room temperature for 3 days to produce a crude extract; (b) suspending the crude extract in water to give a water extract, filtrating the water extract, concentrating the filtrate at 70° C. with a concentrator, adjusting the pH of the concentrate to 3.3˜3.7, preferably to 3.4˜3.6 and most preferably to 3.5 with 10% HCl; (c) adding hexane to the aqueous extract to remove other organics and dividing the aqueous layer into an ethyl acetate (EtOAc) extract and an aqueous extract; (d) adjusting the pH of the aqueous extract to 4.3˜4.7, preferably to 4.4˜4.6 and most preferably to 4.5, washing the aqueous extract with buthanol (BuOH), removing the solvent at 70° C. in a concentrator, drying the residue in a vacuum for 5 hours to afford pure magnesium lithospermate B as a yellowish brown powder; and (e) adding distilled water to the ethyl acetate (EtOAc) extract, adjusting the pH of the solution to 4.3˜4.7, preferably to 4.4˜4.6 and most preferably to 4.5 with 10% HCl, and performing extraction with fresh ethyl acetate, concentration and drying to further obtain magnesium lithospermate B.

In another aspect thereof, the present invention provides a method for producing a Salviae miltiorrhizae extract containing a high concentration of magnesium lithospermate B, comprising (a) finely cutting roots of Salviae miltiorrhizae into sections 1 cm long, immersing the sections in distilled water, refluxing the root sections in distilled water at 100° C. for 2 hours in a water bath to produce an aqueous extract; (b) concentrating the aqueous extract into a one fourth volume at 70° C. in a concentrator, adding acetone to the concentrate, stirring the solution for 30 min, and filtering the precipitates; (c) concentrating the filtrate at 70° C. in a concentrator to remove the acetone, adding fresh acetone to the concentrate, stirring the solution for 30 min and filtering precipitates; (d) concentrating the filtrate in a concentrator at 70° C. to remove the acetone, adjusting the pH of the acetone-free concentrate to 0.8˜1.2, preferably to 0.9˜1.1 and most preferably to 1, and extracting the concentrate two or three times with water-saturated buthanol (BuOH); and (e) concentrating the extract at 90° C. in a concentrator, drying the concentrate in a vacuum for 5 hours to obtain a high concentration of a Salviae miltiorrhizae extract.

A better understanding of the present invention may be obtained in light of the following examples, which are set forth to illustrate, but are not to be construed to limit the present invention. Therefore, the present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced other than as specifically described

### Example 1

1 kg of roots of Salviae miltiorrhizae was finely cut into pieces about 1 cm in size. These root pieces were immersed in 8 liters of 80% methanol (MeOH) at room temperature for 3 days to provide a crude extract. The crude extract was suspended in water and the suspension was filtered and concentrated into 2 liters at 70° C. in a concentrator, followed by adjusting the pH of the aqueous extract to 3.5 with 10% HCl. The aqueous extract concentrate was washed with hexane (ll×3) to remove other organics and then with ethyl acetate (EtOAc) (1 l×3) to give an ethyl acetate (EtOAc) extract and an aqueous extract. After the pH of the aqueous solution was adjusted to 4.5 with 10% HCl, the aqueous solution was extracted with butanol (BuOH) (2 l×3) and the extract thus obtained was concentrated at 70° C. in a concentrator to remove the solvent. Drying the concentrate in a vacuum for 5 hours produced 18 g of pure magnesium lithospermate B as a yellowish brown powder. The ethyl acetate (EtOAc) extract was diluted in 1 l of distilled water and adjusted to pH 4.5 with 10% HCl, followed by extraction with fresh ethyl acetate (1 l), concentration and drying to further produce 2 g of magnesium lithospermate B.

In comparison to the isolation of magnesium lithospermate B from Salviae miltiorrhizae through column chromatography using the expensive resin MCI gel CHP 20P or Sephadex LH-20 in a stationary phase, as described by Takashi Tanaka, et al., in “Magnesium and Ammonium-Potassium Lithospermate B, the Active Principles Having a Uremia-Preventive Effect from Salviae miltiorrhizae,” Chem. Pharm. Bull. 37(2) 340-344 (1989) (Comparative Example 1) and through column chromatography using silica gel (which is inexpensive compared to Sephadex LH-20 and MCI gel CHP 20P, but still relatively expensive) as an absorbent (stationary phase) (Comparative Example 2), the isolation through the procedure of Example 1 resulted in the production of magnesium lithospermate B at 2 fold higher yield (Table 1, below).

TABLE 1

| | Ex. 1 | C. Ex. 1 | C. Ex. 2 |
|---|---|---|---|
| Salviae miltiorrhizae (kg) | 1 | 1 | 1 |
| Isolation means | | MCI gel and | Silica |
| | | Sephadex resin | gel |
| Isolated Mg lithospermate B | 20 | 11.86 | 11 |
| (g) | | | |
| Yield (%) | 2 | 1 | 1 |

### Example 2

200 g of Salviae miltiorrhizae roots was cut into pieces about 1 cm in size, and 800 ml of distilled water was added to the sectioned roots of Salviae miltiorrhizae, followed by refluxing at 100° C. for 2 hours in a water bath to produce an aqueous extract. In a concentrator, 800 ml of the aqueous extract was concentrated to 200 ml at 70° C. The addition of 300 ml of acetone was followed by stirring for 30 min and then filtration of the precipitates. At 70° C., 300 ml of acetone was removed to concentrate 500 ml of the filtrate to 200 ml. Again, 300 ml of acetone was added to the concentrate, which was then stirred for 30 min before the precipitates were filtered out. At 70° C., 500 ml of the filtrate was concentrated to 200 ml to remove 300 ml of acetone. The pH of 200 ml of the aqueous extract was adjusted to 1, followed by washing two or three times with 200 ml of water-saturated buthanol (BuOH). The extract was concentrated at 90° C. in a concentrator and dried in a vacuum to obtain 9.144 g of a highly concentrated Salviae miltiorrhizae extract. In 9.144 g of the highly concentrated Salviae miltiorrhizae extract, the amount of magnesium lithospermate B was measured to weigh 5.486 g (60%).

In comparison to the production of a Salviae miltiorrhizae extract containing magnesium lithospermate B from Salviae miltiorrhizae using 80% methanol (MeOH) as a separation solvent, as described by Mankil Jung, et al., in “Effective Isolation of Magnesium Lithospermate B and Its Inhibition of Aldose Reductase and Fibronectin on Mesangial Cell Line,” Chem. Pharm. Bull. 50(8) 1135-1136 (2002) (Comparative Example 3), the method described in Example 2 produced a Salviae miltiorrhizae extract which contained magnesium lithospermate B at a 16-fold higher concentration (Table 2, below). When formulated into a medicine, this extract, containing a high concentration of magnesium lithospermate B, enjoys an advantage of enabling a significant decrease in the daily dosage.

TABLE 2

| | Ex. 2 | C. Ex. 3 |
|---|---|---|
| Salviae miltiorrhizae (kg) | 1 | 1 |
| Separation Solvent | Distilled | 80% |
| | water/BuOH | Methanol |
| Mg lithospermate B/Salviae | 27.45/45 | 11/300 |
| miltiorrhizae Extract (g) | | |
| Content of Mg lithospermate B | 60 | 3.7 |
| in Salviae miltiorrhizae | | |
| extract (%) | | |

### INDUSTRIAL APPLICABILITY

As described hitherto, a Salviae miltiorrhizae extract containing magnesium lithospermate B (MLB), which is an active ingredient highly useful in the treatment and prevention of diabetic nephropathy, microalbuminuria and uremia, can be produced easily and economically on a mass scale by the method according to the present invention.

1. A method for isolating magnesium lithospermate B from Salviae miltiorrhizae, comprising:
(a) finely cutting roots of Salviae miltiorrhizae into sections 1 cm in size and immersing the sections in 80% methanol at room temperature for 3 days to produce a crude extract;
(b) suspending the crude extract in water to give a water extract, filtrating the water extract, concentrating the filtrate at 70° C. with a concentrator, and adjusting the pH of the concentrate to 3.3˜3.7 with 10% HCl;
(c) adding hexane to the aqueous extract to remove other organics and dividing the aqueous layer into an ethyl acetate (EtOAc) extract and an aqueous extract;
(d) adjusting the pH of the aqueous extract to 4.3˜4.7, washing the aqueous extract with buthanol (BuOH), removing the solvent at 70° C. in a concentrator, and drying the residue in a vacuum for 5 hours to afford pure magnesium lithospermate B as a yellowish brown powder; and
(e) adding distilled water to the ethyl acetate (EtOAc) extract, adjusting the pH of the solution to 4.3˜4.7 with 10% HCl, and performing extraction with fresh ethyl acetate, concentration and drying to further obtain magnesium lithospermate B.

2. The method according to claim 1, wherein the pH of the concentrate in step (b) is adjusted to 3.4˜3.6.

3. The method according to claim 1, wherein the pH of the aqueous extract in step (d) is adjusted to 4.4˜4.6 and the pH of the ethyl acetate (EtOAc) in step (e) solution is adjusted to 4.4˜4.6.

4. The method according to claim 1, wherein magnesium lithospermate B is produced from roots of Salviae miltiorrhizae at a yield of 2% or higher.

5. A method for producing a Salviae miltiorrhizae extract containing a high concentration of magnesium lithospermate B, comprising:
(a) finely cutting roots of Salviae miltiorrhizae into sections 1 cm long, immersing the sections in distilled water, and refluxing the root sections in distilled water at 100° C. for 2 hours in a water bath to produce an aqueous extract;
(b) concentrating the aqueous extract to one fourth its original volume at 70° C. in a concentrator, adding acetone to the concentrate, stirring the solution for 30 min, and filtering out precipitates;
(c) concentrating the filtrate at 70° C. in a concentrator to remove the acetone, adding fresh acetone to the concentrate, stirring the solution for 30 min and filtering out precipitates;
(d) concentrating the filtrate in a concentrator at 70° C. to remove the acetone, adjusting the pH of the acetone-free concentrate to 0.8˜1.2 and extracting the concentrate two or three times with water-saturated buthanol (BuOH); and
(e) concentrating the extract at 90° C. in a concentrator and drying the concentrate in a vacuum for 5 hours to obtain a high concentration of a Salviae miltiorrhizae extract.

6. The method according to claim 5, wherein the pH of the acetone-free concentrate is adjusted to 0.9˜1.1.

7. The method according to claim 5, wherein the Salviae miltiorrhizae extract contains magnesium lithospermate B at a concentration of 60% or higher.
